(19) United States
(12) Patent Application Publication
(10) Pub. No.: US 2005/0009914 A1
(43) Pub. Date: Jan. 13, 2005

(54) Use of a cysteine-containing substance to increase the ventilatory activity and erythropoientin production

(76) Inventor: Wulf Droge, Heidelberg (DE)

Correspondence Address: BROMBERG & SUNSTEIN LLP, 125 SUMMER STREET, BOSTON, MA, 02110-1618, US

(21) Appl. No.: 10/498,393

(22) PCT Filed: Dec. 10, 2002
(86) PCT No.: PCT/CA02/01900

(30) Foreign Application Priority Data
Dec. 11, 2001 (DE) 101 60 796.2

(51) Int. Cl. A61K 31/198

(52) U.S. Cl. 514/562

(57) ABSTRACT

The said invention deals with the use of cysteine, a cysteine precursor substance or a cysteine derivative or a cysteine-containing substance to increase the oxygen supply to the tissue by increasing the ventilatory activity and/or by increasing the production of the blood-forming hormone erythropoietin (EPO), especially for the treatment of aging-related or disease-related conditions associated with a decreased oxygen supply to the tissue, especially in elderly subjects, in the treatment of malignant diseases and in cardiorespiratory diseases.

The invention deals with the use of cysteine, a cysteine precursor, or a cysteine derivative or a cysteine-containing substance to improve the oxygen delivery to the tissue by increasing the ventilatory response and the production of the blood-forming hormone erythropoietin (EPO), especially for the treatment of aging-related or disease-related conditions associated with a decreased oxygen supply to the tissue.

The oxygen concentration of the tissue plays an important physiological role in all higher organisms. The maintenance of an adequate oxygen supply is mainly ensured by an adequate number of erythrocytes in the blood and by the ventilatory activity. In the young healthy organism, any decrease in arterial oxygen concentration (hypoxia) activates the oxygen-sensing arterial chemoreceptors and stimulates in addition the EPO production in liver and kidney cells.

Although the said mechanisms are able to ensure under normal conditions the adequate oxygen supply to the tissue, the oxygen supply decreases in old age, malignant diseases and in cardiorespiratory diseases.

These conditions are presently being treated with synthetic EPO which is typically produced by recombinant DNA technology or with other drugs that increase the ventilatory activity. However, the presently available treatment procedures have substantial disadvantages such as high costs and undesirable side-effects. In addition, the available drugs improve typically either the ventilatory activity or the EPO concentration in the plasma but not both simultaneously.

It is the purpose of this invention to provide an alternative agent for the production of a drug to increase the ventilatory response and EPO concentration, especially in old age and certain disease conditions such as malignant or cardiorespiratory diseases. Preferred is a “physiological” agent which produces little or no side-effects.

The invention solves this problem by providing such an alternative agent based on the use of cysteine or a cysteine derivative to increase the oxygen supply to the tissue, the ventilatory response and the EPO concentration in the plasma.

Cysteine, its biochemical precursors, cysteine derivatives, and cysteine-containing substances such as cysteine-containing proteins are typically “physiological” agents which play normally a role in the natural metabolism. Accordingly, there are so far no significant, side-effects known for the treatment with cysteine or other cysteine derivatives, especially N-acetylcysteine.

By treatment with cysteine, a cysteine derivative, a cysteine precursor, or a cysteine-containing substance, the plasma thiol/disulfide redox status will be shifted to a more reducing condition. This in turn will increase the hypoxic ventilatory activity and the EPO concentration in the plasma of the patients. Ultimately, this treatment improves the oxygen supply to the tissues.

These effects of the cysteine derivative N-acetylcysteine (NAC) on the hypoxic ventilatory activity and the plasma EPO concentration, which implicate ultimately the improvement of the oxygen supply to the tissue, was shown in the context of this invention through a placebo-controlled double-blind clinical trial.

Because the thiol group (SH group) of NAC is responsible for the antioxidative shift of the plasma thiol/disulfide redox state, it is reasonable to be expected that the said effects of NAC on the ventialatory activity and the plasma EPO concentration and oxygen supply to the tissue is also provided by other physiological thiol-containing substances, such as cysteine itself, physiological precursor substances of cysteine, other cysteine derivatives, or cysteine-containing polypeptides.

The general term “cysteine derivative” will therefore be used here to describe all substituted cysteine derivatives, physiological cysteine precursors, functional thiol groups, especially N-acetylcysteine as well as cysteine-containing substances, such as cysteine-containing proteins or peptides.

The mechanism of the oxygen sensors which regulate the ventilatory response and the production of EPO is not completely understood (Semenza G L, “Perspectives on oxygen-sensing”; Cell, 1999; 98; 281-284). There are, however, several indications that the production of the superoxide anion radical may serve as a second messenger at least in some oxygen sensors. The superoxide anion, in turn,: may activate various redox-sensitive signaling pathways (Acker et al., “Mechanisms Of O₂ sensing in the carotid body in comparison with other O₂-sensing cells”; NIPS; 1955; 10; 211-216; Acker et al., “Indications for an NADPH oxidase as a possible pO₂-sensor in the rat carotid body”; FEBS Lett.; 1989; 256; 75-78; Prabhakar et al., “Oxygen sensing by the carotid body chemoreceptor”; J. Appl. Physiol.; 2000; 88; 2287-2295; Fandrey et al., “Role of hydrogen peroxide in hypoxia-induced erythropoietin production”; Biochem J.; 1994; 303; 507-510; Huang et al., “Activation of hypoxia-inducible transcription factor depends primarily upon redox-sensitive stabilization of its α-subunit”; J. Biol. Chem.; 1996; 271; 32253-32259).

Whereas it was previously known that the oxygen sensors are responsive to changes in the oxygen concentration, the invention is based on the hypothesis that these oxygen sensors are redox sensors which also respond to changes in the plasma thiol/disulfide redox status.

In the context of the placebo-controlled double-blind study of two randomly selected groups of subjects, it was shown that the administration of N-acetylcysteine (NAC), a substance which is known to increase the plasma thiol level and/or plasma thiol/disulfide redox status (thiol² disulfide¹), causes unexpectedly an increase in ventilatory activity and plasma erythropietin concentration.

The placebo-controlled double-blind study was performed with healthy, normotonic male non-smoking subjects. The subjects had not taken alcohol, caffeine, or any medication at least during the last 24 hours prior to the study. Also, they had not been engaged in extensive physical exercise for at least 12 hours prior to the study. Twenty-eight subjects were recruited into the study and randomized for the treatment with NAC or placebo. NAC (200 mg capsules) or placebo were administered in three doses of 600 mg per day at 8.00 a.m., 4.00 p.m. and 11.00 p.m.

The plasma concentrations of acid soluble thiol (mainly cysteine) and cystine (cysteine disulfide) were determined in the cubital venous blood by a photometric assay and by the amino acid analyzer, respectively. The plasma thiol/disulfide redox status of the subjects was computed on the basis of these data according to the equation: redox status=thiol² disulfide⁻¹ (μM). The EPO concentration in serum was determined by the help of a commercial EPO ELISA kit.

In addition, the hypoxic ventilatory response of the subjects was quantitatively determined. The ventilatory activity was determined under two different experimental conditions using an equipment of oxychon-beta. The first measurement, the so-called “isocapnic hypoxic ventilatory response” (isocapnic HVR), was tested under normoxic conditions, i.e., under conditions of normal oxygen concentration and constant carbondioxide concentration. The second measurement, the so-called poikilocapnic hypoxic ventilatory response (poikilocapnic HVR), was determined under normobaric conditions in a hypoxia chamber with a constant FiO₂ of 12%.

The term “poikilocapnic” describes study conditions characterized by a variable carbondioxide concentration in the exspiratory air, whereas the term “isocapnic” refers to conditions with a constant carbondioxide concentration.

The results of this study are shown in the following figures:

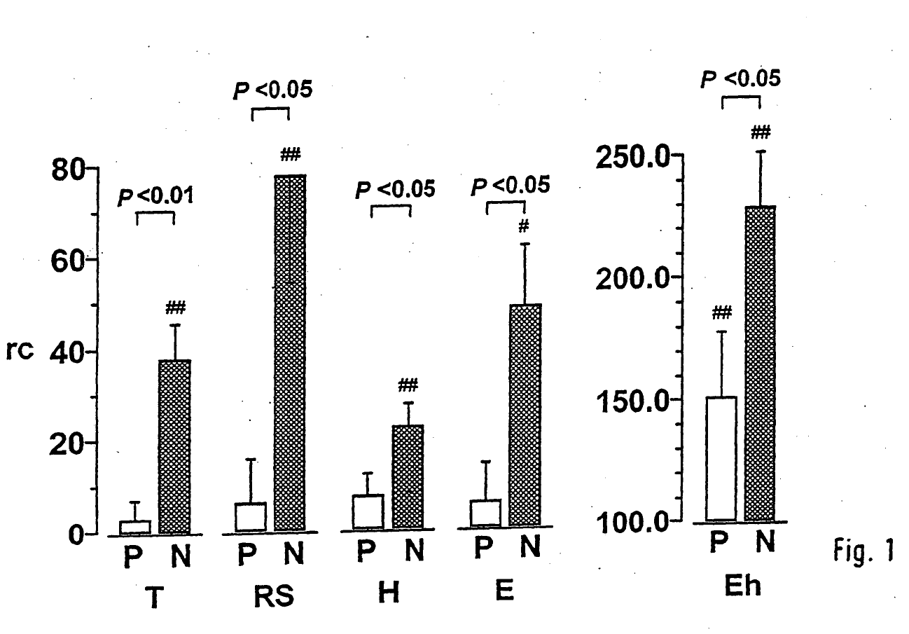

FIG. 1 shows the relative change (rc as %) in plasma thiol concentration (T), plasma redox status (RS), isocapnic hypoxic ventilatory reponse (H), plasma EPO concentration (E), and plasma EPO concentration 2 hrs after the 6-hr-exposure to the hypoxia chamber (Eh). The significance for the differences between N-acetylcysteine-treated group (N) and the placebo-treated control group (P) is indicated by its P-value.

FIG. 1 shows accordingly that the NAC-treated group had not only a significantly increased plasma thiol concentration (P<0.01) and increased plasma thiol/disulfide redox status (thiol² disulfide⁻¹) (P<0.05) but also a significantly increased isocapnic hypoxic ventilatory response as a representative indicator of the ventilatory activity and an increased plasma EPO concentration before and after exposure to the hypoxia chamber (again P<0.05) if compared to the placebo-treated group.

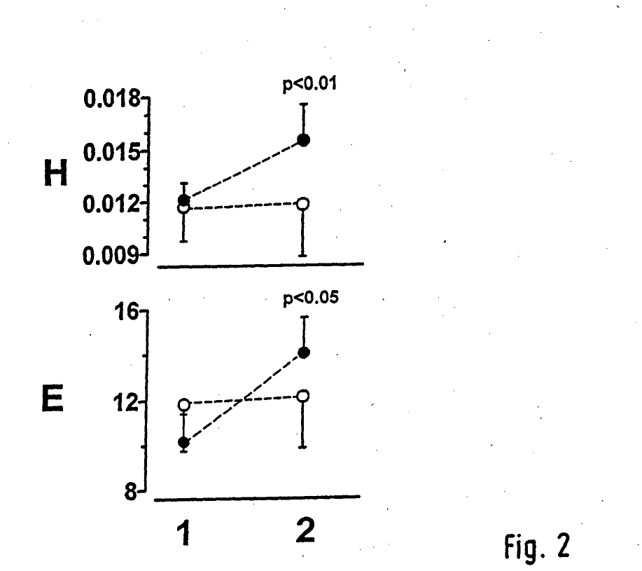

FIG. 2 shows the absolute values of the isocapnic hypoxic ventilatory responses (H) and the plasma EPO concentrations (E) before (time 1) as well as 4 and 5 days, respectively, after start of the treatment (time 2).

The increase in ventilatory activity (H) and the plasma EPO concentration (E) between time 1 and time 2, which is seen in the N-acetylcysteine-treated group (closed symbols), is significantly different from that of the placebo-treated control group (open symbols) (P value <0.01 and <0.05, respectively).

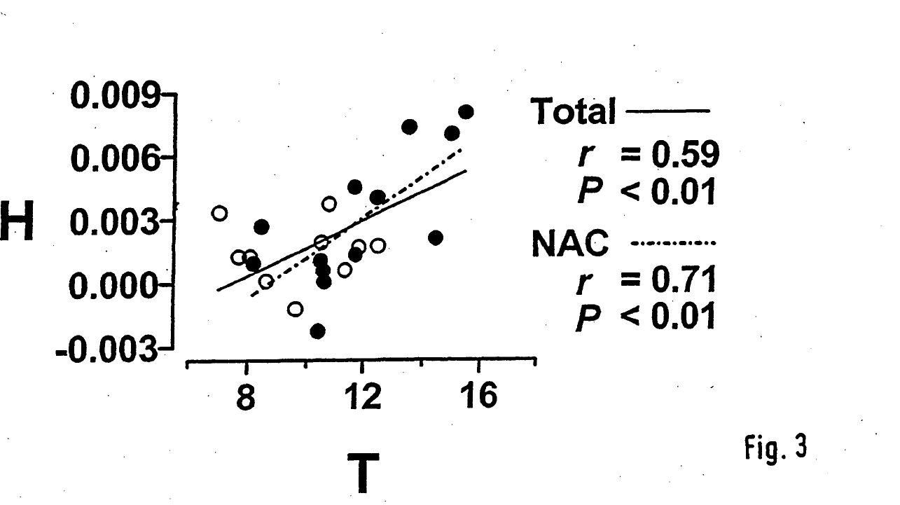

FIG. 3 shows the correlation between the individual values of the poikilocapnic hypoxic ventilatory responses H (units are min⁻¹%⁻¹ kg⁻¹) at the end of the 6-hr-exposure to the hypoxia chamber 5 days after the start of N-acetylcysteine treatment (black symbols) or placebo treatment (open symbols) and the mean plasma thiol concentration T (μM) of the corresponding subjects.

The high values of the correlation coefficient (r) as well as the small P-values demonstrate the significance of the correlation between the respiratory activity, i.e., the poikilocapnic hypoxic ventilatory response, and the mean plasma thiol concentration of the subject (r=0.59 and P<0.01) for the combined treatment groups r=0.71 and P<0.01 for the N-acetylcysteine group.

The invention deals therefore with the use of cysteine or a cysteine derivative for the preparation of a medication designed to increase the oxygen supply to the tissue, the ventilatory activity, and the plasma EPO concentration.

A preferred application of the invention is the use of cysteine or a cysteine derivative for the preparation of a medication for the enhancement of the respiratory activity and the concomitant increase in plasma erythropoietin concentration.

The oxygen concentration in the tissue which is determined by both the ventilatory activity and the plasma erythropoietin concentration is abnormally decreased in certain conditions especially in elderly subjects, in cancer patients, or in cardiorespiratory diseases.

One of the preferred applications of the invention deals therefore with the use of cysteine or a cysteine derivative in the preparation of a medication for the treatment of conditions with abnormally decreased oxygen supply especially in elderly subjects, malignant diseases, especially in the advanced stages of malignant diseases, and in cardiorespiratory diseases.

The use of N-acetylcysteine in the preparation of a medication for the increase in of the oxygen supply to the tissue, ventilatory activity, and plasma EPO concentration is preferred.

Cysteine or a cysteine derivative may also be applied in combination with other substances and agents which increase the oxygen supply in the tissue, the respiratory activity or the plasma EPO concentration. Cysteine or one of the cysteine derivatives may be administered either as a powder, as a solution, as a tablet, or as a “slow release tablet”. The formulation may also contain conventional additives including substances which improve the taste, stabilizing agents, antioxidants, or similar additives. Cysteine or the cysteine derivative is typically applied orally. However, it may alternatively applied by any other practical method, especially by parenteral or intravenous injection.

Cysteine or a cysteine derivative, especially N-acetylcysteine, is typically applied for the increase in oxygen supply to the tissue, ventilatory activity and plasma EPO concentration at daily doses between 0.1 g and 20 g, preferentially in daily doses between 0.1 g and 10 g, more preferentially in daily doses between 0.4 g and 3 g. However, the optimal dosis depends on the plasma thiol concentration and/or the plasma thiol/disulfide redox status of the individual patient and should be chosen accordingly.

Another formulation of the invention includes therefore the use of a pharmaceutic composition, containing cysteine or a cysteine derivative for the preparation of a medication for the increase in the oxygen supply to the tissue, ventilatory activity and/or plasma EPO concentration.

The invention refers also to the use of cysteine or a cysteine derivative for the increase in the oxygen supply to the tissue, in respiratory activity and plasma EPO concentration, as well as the simultaneous increase in ventilatory activity and plasma EPO concentration, the use of the cysteine derivative, N-acetylcysteine, being preferred. The use of cysteine or a cysteine derivative is furthermore preferred for the treatment of conditions with a decreased oxygen supply, especially in elderly subjects, in malignant diseases, or in cardiorespiratory diseases. The use of a pharmaceutic composition containing cysteine or a cysteine derivative for the increase in the oxygen supply, the ventilatory activity and/or the plasma EPO concentration is also preferred.

1. Use of cysteine or a cysteine derivative for the preparation of a medication to increase the oxygen supply in the tissue.

2. Use of cysteine or a cysteine derivative for the preparation of a medication to increase the ventilatory activity.

3. A method of using a cysteine or a cysteine derivative for the preparation of a medication to increase the plasma erythropoietin concentration.

4. A method of using a cysteine or a cysteine derivative for the preparation of a medication that simultaneously increases the ventilatory activity and the plasma erythropoietin concentration.

5. A method of using a cysteine or a cysteine derivative for the preparation of a medication according to any one of the claims 1-4 for the treatment of conditions with decreased oxygen supply.

6. A method of using a cysteine or a cysteine derivative to increase the oxygen supply in the tissue.

7. A method of using a cysteine or a cysteine derivative to increase the ventilatory activity.

8. A method of using a cysteine or a cysteine derivative to increase the plasma erythropoietin concentration.

9. A method of using a cysteine or a cysteine derivative to simultaneously increase the ventilatory activity and the plasma erythropoietin concentration.

10. A method of using a cysteine or a cysteine derivative according to any one of the claims 6-9 for the treatment of conditions with decreased oxygen supply.

11. A method of using a cysteine or a cysteine derivative according to claim 10 in elderly subjects.

12. A method of using a cysteine or a cysteine derivative according to claim 10 in malignant diseases.

13. A method of using a cysteine or a cysteine derivative according to claim 10 in cardiorespiratory diseases.

14. A method according to any one of the claims 1-4 or 6-9 wherein the cysteine derivative is N-acetylcysteine.

15. A method of using a pharmaceutical composition containing cysteine or a cysteine derivative according to any one of the claims 1-4 or 6-9.

16. A method according to claim 5 wherein the cysteine derivative is N-acetylcysteine.

17. A method according to claim 10 wherein the cysteine derivative is N-acetylcysteine.

18. A method of using a pharmaceutical composition containing cysteine or a cysteine derivative according to any of claims 4 or 9.

19. A method according to claim 13 wherein the cysteine derivative is N-acetylcysteine.

20. A method of using a pharmaceutical composition containing cysteine or a cysteine derivative according to claim 13.
